(19) United States
(12) Patent Application Publication
(10) Pub. No.: US 2022/0304989 A1
(43) Pub. Date: Sep. 29, 2022

(54) APPLICATION OF ALKALOID COMPOUND HIP IN TREATMENT OF CARDIAC TRAUMA

(71) Applicant: HUZHOU COLLEGE, Huzhou City (CN)

(72) Inventors: Wenna ZHOU, Huzhou City (CN); Honglun WANG, Huzhou City (CN); Jian OUYANG, Huzhou City (CN)

(21) Appl. No.: 17/654,935

(22) Filed: Mar. 15, 2022

(30) Foreign Application Priority Data
Mar. 29, 2021 (CN) 202110334650.X

(51) Int. Cl. A61K 31/437; A61P 9/04; A61K 9/20; A61K 9/00; A61K 9/08; A61K 47/26; A61K 47/02

(52) U.S. Cl. CPC A61K 31/437; A61P 9/04; A61K 9/2018; A61K 9/2059; A61K 9/2054; A61K 9/2013; A61K 9/2009; A61K 9/0019; A61K 9/08; A61K 47/26; A61K 47/02

(57) ABSTRACT

The disclosure provides a use of an alkaloid compound Hip in treatment and/or prevention of cardiac trauma. Upon studying activity of the alkaloid compound Hip, it is verified that the alkaloid compound Hip provided in the disclosure can inhibit apoptosis induced by cardiomyocyte toxicity, through down-regulating expression levels of p-JNK and/or Cleaved-Caspase-3 to inhibit anthracycline DOX-induced cardiomyocyte apoptosis, thereby alleviating cardiomyocyte toxicity to achieve treatment and/or prevention of cardiomyocyte toxicity. Thus, the alkaloid compound Hip can be used to manufacture a medicine for treatment and/or prevention of myocardial injury-related diseases.

### TECHNICAL FIELD

The disclosure relates to the field of medical application of an alkaloid compound.

### BACKGROUND

Seabuckthorn (Hippophae rhamnoides linn.) is a deciduous shrub of seabuckthorn genus in elaeagaceae, characterized by drought and sand resistance, and can survive on saline-alkali land, so it is widely used in soil and water conservation. Sea-buckthorn is planted in large quantities in northwestern China for desert greening.

Seabuckthorn is rich in nutrients, containing active substances such as a variety of vitamins, flavonoids, triterpenoids, oils and fatty acids, phenols, volatile oils, trace elements, phospholipids, and serotonin, and various amino acids and proteins necessary for the human body.

Seabuckthorn seeds are often used to extract oil to make seabuckthorn oil. The seabuckthorn oil contains 206 kinds of active substances that are beneficial to the human body, including 46 kinds of biologically active substances, and a large amount of vitamin E, vitamin A, flavonoids, etc. After oil extraction, a large amount of seabuckthorn seed meal will be produced, which is often discarded as wastes, or produced as dry powder as feeds with very low price. If more use value of seabuckthorn seed meal can be developed, it will have great economic significance.

In addition, there are still many active substances in seabuckthorn that have not been isolated and identified.

If more in-depth and subtle research and activity mechanism discussion will be performed on new chemical ingredients and pharmacological effects of seabuckthorn, it is expected to develop more safe and effective natural plant-based medicines for treatment of diseases.

### SUMMARY

At present, many medicines may bring about cardiotoxic side effects during treatment, especially during chemotherapy of malignant tumors in which the cytostatic agent used can cause severe cardiomyocyte toxicity and have a negative impact on prognosis and recovery of patient.

It has been reported that anthracyclines are generally cardiotoxic, and widely used in clinical practice due to good antitumor activity. Anthracyclines includes doxorubicin, daunorubicin, aclacinomycin, epirubicin, pirarubicin, idarubicin and mitoxantrone. Doxorubicin (formerly known as adriamycin) is one of broad spectrum antitumor medicines commonly used clinically and has achieved good curative effect in treatment of malignant tumors, and has been widely used in treatment of tumors such as solid tumors, leukemia, lymphoma, and breast cancer since the early 1960s. It has a high affinity for myocardial tissue, and its main anti-tumor mechanism is in that its anthracycline plane can be directly embedded between DNA base pairs, thereby interfering with the transcription process, preventing mRNA synthesis, and inhibiting both DNA and RNA synthesis. Therefore, it has an effect during all stages of the cell cycle and belongs to the non-specific medicine for the cell cycle. However, accumulation and dose-dependent cardiomyocyte toxicity of doxorubicin has been a major problem in tumor therapy from medicine development to clinical application, which are incidence of congestive heart failure induced at the therapeutic dose of more than 550 mg/m² of up to 11%˜30% and fatality rate of up to 50%˜60%. The mechanism of doxorubicin-induced cardiomyopathy is still not fully understood, but different from its antitumor mechanism. Many studies have shown that doxorubicin can induce oxidative stress, calcium overload, and mitochondrial damage of myocardial cells, and inhibit specific gene expression in myocardial cells, thereby inducing apoptosis and necrosis of myocardial cells as well as myocardial fibrosis, reducing myocardial contractility, and eventually leading to ventricular remodeling and congestive heart failure.

Therefore, it is of great significance to develop anti-cardiotoxic substances that can reduce damage of cardiotoxic substances to heart.

In view of the above problems, the disclosure provides a use of a natural compound of indole alkaloid Hip (Hippophamide) extracted from seabuckthorn seed meal in the treatment and/or prevention of cardiac trauma. The compound Hip can be used for prevention and/or treatment of cardiac trauma by down-regulating expression levels of p-JNK and/or Cleaved-Caspase-3 to inhibit anthracycline DOX-induced cardiomyocyte apoptosis and alleviate cardiomyocyte toxicity, thereby achieving treatment and/or prevention of cardiomyocyte toxicity. Thus, the compound Hip can be used to manufacture products for treatment and/or prevention of myocardial injury-related diseases.

The disclosure provides the use of the alkaloid compound Hip having a structural formula in treatment and/or prevention of cardiac trauma, the structural formula being:

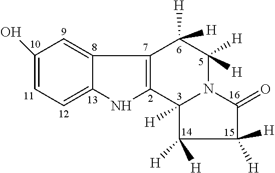

Further, the cardiac trauma is selected from cardiomyocyte toxicity and/or cardiomyocyte apoptosis.

The cardiac trauma described in the use in the disclosure is caused by anthracyclines.

Further, the anthracyclines are selected from one or more of doxorubicin, daunorubicin, aclacinomycin, epirubicin, pirarubicin, idarubicin, and mitoxantrone.

Further, the use is to alleviate and/or inhibit cardiomyocyte poptosis.

Further, the use is to down-regulate the expression levels of Cleaved-Caspase-3 and/or p-JNK.

Further, the use is as a Cleaved-Caspase-3 inhibitor and/or a p-JNK inhibitor.

The Cleaved-Caspase-3 inhibitor refers to a product that can reduce an amount of Cleaved-Caspase-3 in body. The p-JNK inhibitor refers to a product that can reduce a amount of p-JNK in body.

The compound in the disclosure, when is used, further comprises a pharmaceutically acceptable auxiliary material. The “Pharmaceutically acceptable auxiliary material” is a general term for all additional materials except for a main medicine in a medicine. The auxiliary material should have the following properties: (1) it has no toxic effect on human body and has almost no side effect; (2) it has stable chemical properties and is not easily affected by a temperature, pH, storage time, etc.; (3) it has no incompatibility with the main medicine, and does not affect efficacy and quality inspection of the main medicine; and (4) it does not interact with packaging materials.

The auxiliary material in the disclosure includes but are not limited to a filler (a diluent), a lubricant (a glidant or anti-adherent), a dispersant, a wetting agent, an adhesive, a regulator, a solubilizer, antioxidant, a bacteriostatic agent, an emulsifier, a disintegrant, etc. The binder includes a syrup, an acacia, a gelatin, a sorbitol, a tragacanth, a cellulose and its derivatives (such as microcrystalline cellulose, sodium carboxymethyl cellulose, ethyl cellulose or hydroxypropyl methyl cellulose, etc.), a gelatin slurry, a syrup, a starch slurry or a polyvinylpyrrolidone, etc. The filler includes lactose, powdered sugar, dextrin, starch and its derivatives, cellulose and its derivatives, an inorganic calcium salt (such as calcium sulfate, calcium phosphate, calcium hydrophosphate, precipitated calcium carbonate, etc.), sorbitol or glycine, etc. The lubricant includes micropowder silicagel, magnesium stearate, talc, aluminum hydroxide, boric acid, hydrogenated vegetable oil, polyethylene glycol, etc. The disintegrant includes starch and its derivatives (such as sodium carboxymethyl starch, sodium starch glycolate, pregelatinized starch, modified starch, hydroxypropyl starch, corn starch, etc.), polyvinylpyrrolidone or microcrystalline cellulose, etc. The humectant include dodecane sodium bisulfite, water or alcohol, etc. The antioxidant includes sodium sulfite, sodium bisulfite, sodium metabisulfite, dibutyl benzoic acid, etc. The bacteriostatic agent include 0.5% phenol, 0.3% cresol, 0.5% chlorobutanol etc. The regulator includes hydrochloric acid, citric acid, potassium hydroxide (sodium), sodium citrate and buffers (including sodium dihydrogen phosphate and disodium hydrogen phosphate), etc. The emulsifier include polysorbate-80, malic acid Sorbitan, Pluronic F-68, lecithin, soybean lecithin, etc. The solubilizer includes Tween-80, bile, glycerin, etc.

There are no special restrictions on administration routes of the compound or composition in the disclosure. Representative administration routes include (but are not limited to) oral, extra-gastrointestinal (intravenous, intramuscular or subcutaneous), and local administration.

A solid dosage form for oral administration include a capsule, a tablet, a pill, a powder and a granule. In these solid dosage forms, the active compound is mixed with at least one conventional inert auxiliary material (or carrier), such as sodium citrate or dicalcium phosphate, or with (a) a filler or extender, for example, starch, lactose, sucrose, glucose, mannitol and silicic acid; (b) a binder such as hydroxymethylcellulose, alginate, gelatin, polyvinylpyrrolidone, sucrose and acacia; (c) a humectant, such as glycerol; (d) a disintegrant, such as agar, calcium carbonate, potato or tapioca starch, alginic acid, certain complex silicates, and sodium carbonate; (e) the slow solvent, such as paraffin; (f) a absorption accelerator such as quaternary amine; (g) a wetting agent such as cetyl alcohol and glyceryl monostearate; (h) a adsorbent such as kaolin; and (i) a lubricant such as talc, hard Calcium fatty acid, magnesium stearate, solid polyethylene glycol, sodium lauryl sulfate, or mixtures thereof. In capsules, tablets and pills, the dosage form may also contain buffering agents.

The solid dosage form such as a tablet, a dragee, a capsule, a pill and a granule can be manufactured with coating and shell materials such as an enteric coating and other materials well known in the art. They may contain an opacifying agent. Release of the active compound or the compounds in such composition can be delayed in a certain part of the digestive tract. Examples of embedded ingredients that can be employed are polymeric substances and waxes. If necessary, the active compound may also form microcapsules with one or more of the auxiliary materials.

A liquid dosage form for oral administration include a pharmaceutically acceptable emulsion, a solution, a suspension, a syrup or a tincture. In addition to an active compound, the liquid dosage form may contain a inert diluent conventionally employed in the art, such as water or other solvents, a solubilizer and an emulsifier, such as ethanol, isopropanol, ethyl carbonate, ethyl acetate, propylene glycol, 1,3-butanediol, dimethylformamide and oil, especially cottonseed oil, peanut oil, corn germ oil, olive oil, castor oil and sesame oil or mixtures of these substances and the like.

Besides these inert diluents, the composition can also contain ab adjuvant such as a wetting agent, an emulsifying and a suspending agent, a sweetening agent, a flavoring agent and a perfuming agent.

In addition to the active compound, the suspension may contain a suspending agent such as ethoxylated isostearyl alcohol, polyoxyethylene sorbitol and sorbitan ester, microcrystalline cellulose, aluminum methoxide and agar, or mixtures of these substances and the like.

The composition for parenteral injection may comprise a physiologically acceptable sterile aqueous or anhydrous solution, a dispersion, a suspension or an emulsion, and a sterile powder for reconstitution into a sterile injectable solution or dispersion. The suitable aqueous and non-aqueous carrier, diluent, solvent or auxiliary material include water, ethanol, polyol and suitable mixtures thereof.

The dosage form for topical administration of the compound in the disclosure includes an ointment, a powder, a patch, a spray and an inhalant. The active ingredient is mixed under sterile condition with a physiologically acceptable carrier and any preservative, buffer, or propellant that may be required if necessary.

The compound in the disclosure may be used in an injection preparation. The injection preparation is selected from a liquid injection preparation (a water needle), a sterile powder for injection (a powder needle) or a tablet for injection (refers to a moulded tablet or pressed tablet of a medicine made under aseptic operation, which are dissolved with water for subcutaneous injection or intramuscular injection when are used).

The powder for injection contains at least excipients in addition to the above-mentioned compound. The excipients in the disclosure are ingredients that are intentionally added to the medicine and should not have pharmacological properties in the quantities used, which may assist in processing, dissolving or leaching of a medicine, as well as delivery through a targeted medical delivery pathway, or assist in stability.

The excipient in the disclosure can be selected from one or a combination of two or more of carbohydrate, inorganic salt, and polymer. The carbohydrate includes monosaccharide, oligosaccharide or polysaccharide.

In one example in the disclosure, in raw material of the tablet, each 250 mg tablet includes the following ingredients in parts by weight: 5-20 parts of the compound Hip, 100-200 parts of lactose, 10-25 parts of starch, 50-80 parts of microcrystalline cellulose, 1-10 parts of magnesium stearate, and 1-10 parts of talc. The tablet further comprises 10 parts of the compound Hip, 150 parts of lactose, 15 parts of starch, 65 parts of microcrystalline cellulose, 5 parts of magnesium stearate, and 5 parts of talc.

In another example in the disclosure, in raw material of the solution, the solution per milliliter includes the following ingredients in parts by weight: 0.2-1 part of the compound Hip, 30-70 parts of glucose, 6-12 parts of sodium chloride, and 900 to 1000 parts of water. The solution further includes 0.5 parts of the compound Hip, 50 parts of glucose, 9 parts of sodium chloride, and 940.5 parts of water.

The disclosure also provides a use of the alkaloid compound Hip in reducing toxic side effects of anthracyclines. That is, the alkaloid compound Hip is administered simultaneously, separately or in stages during treatment with anthracyclines having a cardiotoxic side effect. For combination administration in the disclosure, a single compound preparation can be manufactured by mixing the alkaloid compound Hip and anthracycline, or the alkaloid compound Hip and anthracycline can be separately manufactured into two preparations and then combined. When two preparations are separately manufactured, the two preparations of the alkaloid compound Hip and the anthracycline can be administered simultaneously or independently. The number and order of administration of the two preparations during separate administration are not limited. The separate administration includes but are not limited to, firstly administering the alkaloid compound Hip and administering the anthracycline preparation after a period of time; or firstly administering the anthracycline preparation and administering the alkaloid compound Hip after a period of time; or firstly administering the alkaloid compound Hip, administering the anthracycline preparation after a period of time and administering the alkaloid compound Hip after a period of time.

The disclosure has the following beneficial effects.

In the disclosure, activity of the alkaloid compound Hip extracted from seabuckthorn seed meal is studied. The results show that the alkaloid compound Hip in the disclosure can inhibit the myocardial cytotoxicity caused by doxorubicin (DOX) through down-regulating expression levels of p-JNK/Cleaved-Caspase-3 to inhibit DOX-induced cardiomyocyte apoptosis, thereby inhibiting doxorubicin (DOX)-induced cardiomyocyte toxicity. Thus, the alkaloid compound Hip can be used to manufacture a product for treatment and/or prevention of cardiac trauma caused by cardiotoxic substance.

### DESCRIPTION OF DRAWINGS

FIG. 1 illustrates a toxicity effect of the alkaloid compound Hip on H9c2 cardiomyocytes;

FIG. 2 illustrates a toxicity effect of the alkaloid compound Hip on DOX-induced H9c2 cardiomyocytes;

FIG. 3 illustrates a toxicity effect of the alkaloid compound Hip on DOX-induced activation of Caspase-3 in H9c2 cardiomyocytes;

FIG. 4 illustrates a toxicity effect of the alkaloid compound Hip on DOX-induced p-JNK/JNK in H9c2 cardiomyocytes.

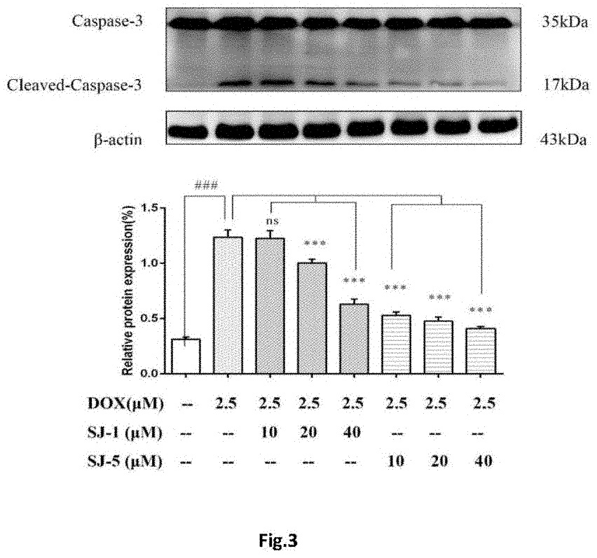

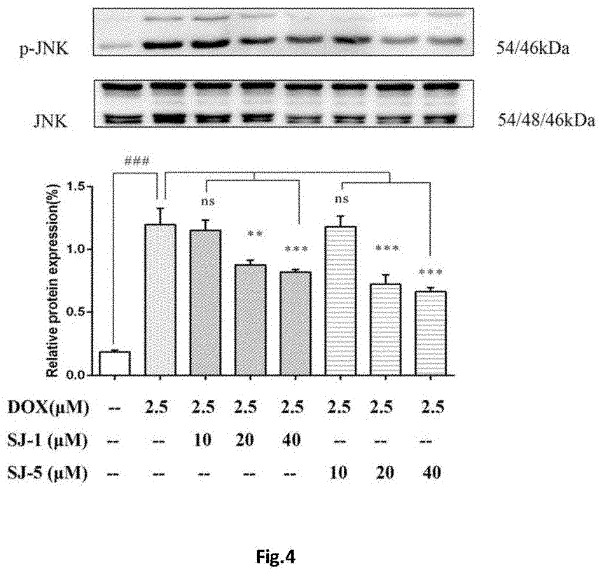

In FIG. 3 and FIG. 4, SJ-5 is an indole alkaloid compound Hip; compared with the Con group, #P<0.05, ##P<0.01, ###P<0.001; compared with the DOX model group, *P<0.05, **P<0.01, ***P<0.001; Mean±SD, n=3.

### DETAILED DESCRIPTION

The technical solutions in the disclosure will be clearly and completely described below. Obviously, the described examples are a part of the embodiments in the disclosure, but not all of the embodiments. Based on the examples in the disclosure, all other examples obtained by those skilled in the art without creative work fall within the protection scope in the disclosure.

The experimental methods in the examples in the disclosure, unless otherwise specified, are conventional methods, and the test materials used, unless otherwise specified, are purchased from conventional biochemical reagent companies, and the data involved in the examples are average values.

The alkaloid compound Hip in the disclosure has the structural formula as follows:

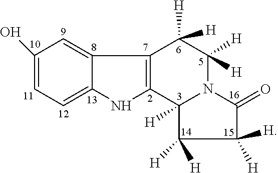

### Example 1. Inhibitory Effect Test of the Compound Hip on Cardiomyocyte Apoptosis in a H9c2 Cardiomyocyte Toxicity Model

Toxicity effect of the alkaloid compound Hip on H9c2 cardiomyocyte

H9c2 cells were incubated with the alkaloid compound Hip at different concentrations (0.1 μM, 1 μM, 5 μM, 10 μM, 20 μM, 40 μM, 80 μM, 160 μM) of for 24 h. The toxicity effect of the alkaloid compound on cardiomyocytes was evaluated by using the MTT colorimetric method.

2. Toxicity effect of the alkaloid compound Hip on DOX-induced H9c2 cardiomyocyte

(1) H9c2 cells were pre-incubated with the alkaloid compound Hip for 1 h, and then added with 2.5 μM doxorubicin (DOX) for 24 h. The effect of the alkaloid compound Hip for DOX-induced cardiomyocyte apoptosis activity was screened by using the MTT colorimetric method. The optimal concentration range for action was determined.

(2) From H9c2 cardiomyocytes which were treated with different tested seabuckthorn alkaloids for 1 h and then treated with 2.5 μM DOX for 24 h, proteins were extract. That is, the H9c2 cardiomyocytes were lysed and scraped, and the cell lysate were collected and centrifuged at 12,000 g for 15 min to collect the supernatant and discard the precipitate. The supernatant was stored at 80° C., and quantified for proteins using the BCA method for later use. SDS-PAGE electrophoresis was performed by using 5% stacking gel (Step1: 80V, 30 min; Step2: 120V, 180 min). The membrane transfer was performed using a PVDF membrane for 30 min at 80 mA. Blocking and hybridization was performed by blocking in 5% nonfat milk powder for 1 h, incubating with a primary antibody overnight and incubating with a secondary antibody at room temperature for 1-2 h with gentle shaking. Development was performed by ECL reagent development. Expression levels of intracellular Caspase-3 and p-JNK/JNK proteins were observed using a gel imaging system.

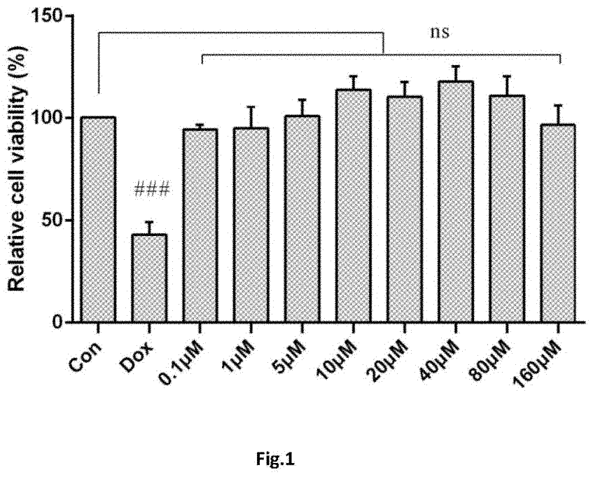

The test results as shown in FIG. 1 shows that the indole alkaloid compound Hip had no statistical difference compared with the Con group within a certain concentration range (0.1˜40 μM), but a significant difference compared with the Con group when the Hip concentration reached 80 μM. This demonstrated that the indole alkaloid compound Hip may have a certain toxic effect on the viability of H9c2 cells when going beyond such concentration.

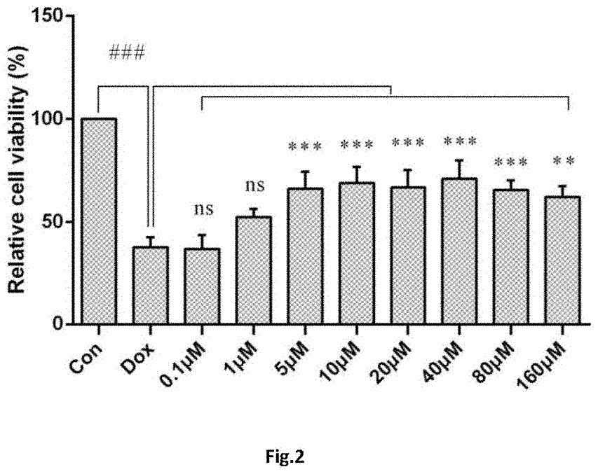

The results in FIG. 2 demonstrated that, the alkaloid compound Hip groups within the different concentration ranges can inhibit DOX-induced myocardial cytotoxicity and is in a certain concentration-dependent manner, compared with the DOX model group. Compared with the model group, the alkaloid compound Hip groups within the concentration range of 5-80 μM were significantly or extremely significant different.

As shown in FIG. 3, compared with the Con group, the DOX model group can significantly increase the expression level of Cleaved-Caspase-3, and compared with the DOX model group, the low, medium and high dose groups of the alkaloid compound Hip (10 μM, 20 μM and 40 μM) can decrease the increased level of Cleaved-Caspase-3 caused by DOX to varying degree, which have an obvious concentration-dependent relationship.

The results shown in FIG. 4 demonstrated that, the DOX model group can significantly increase the expression level of p-JNK compared with the Con group, and there was no statistical difference in the expression level of p-JNK between the low-dose group (10 μM) and the DOX model group of alkaloid compound, but there was extremely significant differences between the middle-dose group (20 μM) and the high-dose group (40 μM), which was obvious concentration-dependent. It is indicated that, the alkaloid compound can down-regulate the expression level of p-JNK in the DOX-induced cardiomyocyte apoptosis model. Combined with analysis of its effect on the expression level of Cleaved-Caspase-3, the alkaloid compound provided by the disclosure can down-regulate the expression levels of p-JNK/Cleaved-Caspase-3 to inhibit DOX-induced cardiomyocyte apoptosis and alleviate the cardiomyocyte toxicity.

To sum up, by administering the tested substances, the alkaloid compound Hip in the disclosure can significantly inhibit the apoptosis induced by cardiomyocyte toxicity and involve down-regulating the expression levels of p-JNK/Cleaved-Caspase-3 to achieve preventive and/or therapeutic effects.

The alkaloid compound Hip in the disclosure can be suitable for use as a medicine for mammals, especially humans, and can be used to prevent and/or treat damage of cardiotoxic doses of medicines and/or cardiac trauma, especially heart failure and change, caused by other chemical substances, such as myocardial toxicity or myocardial fibrosis of anthracyclines.

The alkaloid compound Hip in the disclosure can be used as a medicine, especially for treatment of cardiotoxic side effects of a medicine, or as an adjuvant therapy. Depending on a type of treatment state, a substance used and a administration route used, the alkaloid compound Hip was used by intravenous injection, oral administration, etc, and the amount used can be different and can be varied.

### Example 2

Tablet Containing the Compound Hip

Each tablet is produced with the following ingredients:

| | |
|---|---|
| [61] The compound Hip | 10 mg |
| [62] Lactose | 150 mg |
| [63] Starch | 15 mg |
| [64] Microcrystalline Cellulose | 65 mg |
| [65] Magnesium Stearate | 5 mg |
| [66] Talc | 5 mg |

250 mg tablets were manufactured by mixing, granulating, blending and tableting.

### Example 3

Injection Solution Containing the Compound Hip

The injection solution is produced with the following ingredients per 1 ml:

| | |
|---|---|
| [71] The compound Hip | 0.5 mg |
| [72] Glucose | 50 mg |
| [73] Sodium Chloride | 9 mg |
| [74] Purified water | 940.5 mg |

The above solid substance was dissolved in purified water, and the solution was aseptically filled into 1 ml ampoules.

The above descriptions are only embodiments in the disclosure, and are not intended to limit the protection scope in the disclosure. Any equivalent process or equivalent structure made by using the description in the disclosure, or direct or indirect application in other related technical fields shall be similarly included within the protection scope in the disclosure.

1. A use of an alkaloid compound Hip having a structural formula in treatment of cardiac trauma, the structural formula being:

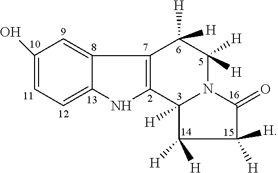

2. The use according to claim 1, wherein the cardiac trauma is selected from cardiomyocyte toxicity and/or cardiomyocyte apoptosis.

3. The use according to claim 1, wherein the cardiac trauma is caused by anthracyclines selected from one or more of doxorubicin, daunorubicin, aclacinomycin, epirubicin, pirarubicin, idarubicin, and mitoxantrone.

4. The use according to claim 3, wherein the cardiac trauma is caused by doxorubicin.

5. The use of the alkaloid compound Hip having a structural formula in alleviation and/or inhibition of cardiomyocyte apoptosis, the structural formula being:

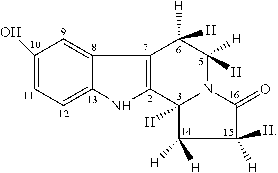

6. A use of an alkaloid compound Hip in reducing toxic side effects of anthracyclines.
